(19) United States
(12) Patent Application Publication
(10) Pub. No.: US 2006/0084125 A1
(43) Pub. Date: Apr. 20, 2006

(54) Methods, devices, and systems for detection of cancer

(76) Inventor: Herzel Laor, Boulder, CO (US)

Correspondence Address: KILPATRICK STOCKTON LLP, 607 14TH STREET, N.W., WASHINGTON, DC, 20005, US

(21) Appl. No.: 11/249,546

(22) Filed: Oct. 14, 2005

Related U.S. Application Data
(60) Provisional application No. 60/619,102, filed on Oct. 18, 2004.

(51) Int. Cl. G01N 33/574

(52) U.S. Cl. 435/7.23; 601/1

(57) ABSTRACT

Disclosed are methods, devices, and systems for early detection of cancer. The cancer may be a glandular cancer, such as breast cancer or prostate cancer. A gland may be made to secrete fluids such that the fluids carry malignant cells that are excreted from the gland or that may be present on the duct walls. Or cells may be dislodged from the surface of an organ for examination. Cells present in the fluid may then be assessed for malignancy. For detection of cancer cells that are present at a low level, the present invention may comprise steps to select for the cells. Using the methods, devices and systems of the present invention may provide for detection of the cancer prior to spread of the disease.

### CROSS REFERENCE TO RELATED APPLICATIONS

The present application claims priority under 35 USC 119(e) from U.S. Provisional Patent Application Ser. No. 60/619,102, filed Oct. 18, 2004. The disclosure of U.S. Provisional Patent Application 60/619,102 is incorporated by reference in its entirety herein.

### FIELD OF INVENTION

The present invention relates to methods, devices and systems for detection of cancer.

### BACKGROUND

There is widespread recognition of the importance of tests for early detection of cancer. In some cases, abnormal or malignant cells exfoliated from the surface of an organ can be identified by cytologic examination of brushings and fluids. For example, a PAP smear may detect abnormal (e.g., pre-cancerous or cancerous) cells of the cervix. Alternatively, genetic abnormalities in cancer cells or pre-cancer cells may be detected using molecular techniques. For example, techniques such as DNA sequence analysis may be used to detect specific mutations and/or structural alterations in DNA. Also, tests for proteins expressed by certain cancers may be performed. For example, screening for prostate-specific antigen (PSA) may be used to identify patients at risk for prostate cancer. Still, PSA screening may suffer from variability of assay methods and a lack of specificity. For example, although malignant prostate cells make higher amounts of PSA, PSA is not specific to cancer cells but is made by both normal and cancerous prostate cells. PSA levels may vary depending upon the age of the patient, the physiology of the prostate, the grade of the cancer, and the sensitivity of PSA levels to pharmacologic agents. Also, the molecular basis for many cancers is as yet unknown, and therefore, molecular tests are not yet comprehensive enough to detect most cancers.

Thus, detection of many cancers still relies on detection of an abnormal mass in the organ of interest. In many cases a tumor is often detected only after a malignancy is advanced and may have metastasized to other organs. For example, breast cancer is typically detected by obtaining a biopsy from a lump detected by a mammogram or by physical examination of the breast. Also, although measurement of prostate-specific antigen (PSA) has significantly improved the detection of prostate cancer, confirmation of prostate cancer typically requires detection of an abnormal morphology or texture of the prostate. Thus, there is a need for methods and devices for earlier detection of cancer.

### SUMMARY

Embodiments of the present invention comprise methods, devices, and systems for detection of cancer. The present invention may be embodied in a variety of ways.

In one embodiment, the present invention may comprise a method for testing a subject for the presence of cancer cells in an organ. The method may comprise the steps of accessing a biological material by causing the biological material to be emitted from the organ, and examining the biological material for at least one molecular characteristic correlated with cancer. In an embodiment, the biological material comprises a plurality of cells. Alternatively, the biological material may comprise parts of cells. In an embodiment, the cells or parts of cells comprise a fluid suspension.

In other embodiments, the present invention may comprise devices and systems for testing a subject for the presence of cancer cells in an organ. For example, in one embodiment, the present invention comprises a device for examining a biological material for at least one molecular characteristic correlated with cancer. In an embodiment, the device comprises a chamber having at least one portion that comprises a focal plane of a viewing device suitable for optical examination of the biological material. In an embodiment, the biological material comprises a plurality of cells. Alternatively, the biological material may comprise parts of cells.

In yet another embodiment, the present invention comprises a system for examining a biological material comprising at least one molecular characteristic correlated with cancer. In an embodiment, the biological material may comprise a plurality of cells. Alternatively, the biological material may comprise parts of cells. Also, in an embodiment, the system may comprise an examination device comprising a chamber having at least one portion that comprises a focal plane of a viewing device suitable for optical examination of the biological material. The system may also comprise a viewing device for optical examination of the biological material in the focal plane. Also, the system may comprise a means to position the biological material in the focal plane. In one embodiment, the means to position the biological material in the focal plan may cause the biological material to move through the focal plane.

There may be advantages associated with certain embodiments of the present invention. In one embodiment, the present invention may allow for early detection of cancer from a gland or other type of organ. For example, by specifically inducing secretion from a gland, cells that are abnormal or malignant may be specifically collected and any abnormal cells detected. Or, collection of a natural body fluid, or a fluid used to dislodge cells from the surface of an organ, may be used to access a biological material of interest.

Also, in various embodiments, the present invention provides systems and devices that may be used to detect abnormal cells regardless of the type of cancer. Thus, the devices and systems of the present invention may be used to monitor cells secreted from breast tissue, pancreatic tissue, adrenal tissue, thyroid tissue, prostate tissue, and other glandular tissues. Or, the devices and systems of the present invention may be used to examine cells that are dislodged or exfoliated from the surface of an organ or a portion of an organ such as the lung, uterus (e.g., cervix), bladder, or the colon.

Further details on each of these aspects of the present invention are set forth in the following description, figures, and claims. It is to be understood that the invention is not limited in its application to the details set forth in the following description, figures and claims, but is capable of other embodiments and of being practiced or carried out in various ways.

### BRIEF DESCRIPTION OF THE FIGURES

FIG. 1 shows a flow-diagram of a method for detection of cancer cells from an organ in accordance with an embodiment of the present invention.

FIG. 2 shows a design of a liquid flow-through test cell in accordance with an embodiment of the present invention where panel A shows a top view; panel B shows a cross-sectional view along the plane of A-A′, and panel C shows an enlargement of a portion of the device.

FIG. 3 shows a system for detection of cancer cells using an in-line flow cell in accordance with an embodiment of the present invention where panel A shows a side view; panel B shows an enlargement of the system shown in panel A, and panel C shows a top view.

FIG. 4 shows a system for detection of cancer cells using an in-line flow cell in accordance with an alternate embodiment of the present invention,

FIG. 5 shows a system for inspection of cells using an in-line flow cell, where panel A shows a flow cell having the focal plane of the objective lens parallel to the flow of the cells in accordance with an embodiment of the present invention, and panel B shows an in-line flow cell having the focal plane of the objective lens angled relative to the flow of the cells in accordance with an embodiment of the present invention.

FIG. 6 shows a test chamber where the liquid is rotated such that cells are forced to the outer portion of the chamber for optical examination in accordance with an embodiment of the present invention.

FIG. 7 shows a system for detection of cancer cells from a gland in accordance with an embodiment of the present invention.

FIG. 8 shows a portion of a system for detection of cancer cells from a gland in accordance with an embodiment of the present invention.

### DETAILED DESCRIPTION

Unless indicated to the contrary, all ranges disclosed herein are to be understood to encompass any and all subranges subsumed therein. For example, a stated range of “1 to 10” should be considered to include any and all subranges between (and inclusive of) the minimum value of 1 and the maximum value of 10; that is, all subranges beginning with a minimum value of 1 or more, e.g. 1 to 6.1, and ending with a maximum value of 10 or less, e.g., 5.5 to 10. Additionally, any reference referred to as being “incorporated herein” is to be understood as being incorporated in its entirety.

It is further noted that, as used in this specification, the singular forms “a,” “an,” and “the” include plural referents unless expressly and unequivocally limited to one referent. The term “or” is used interchangeably with the term “and/or” unless the context clearly indicates otherwise.

As used herein, a “gland” or “glandular tissue” is an organized arrangement of secretory cells. Most glands are organized into secretory units, which are described as either tubules, acini, or cords, depending upon the shape of the secretory unit. A simple gland has an unbranched duct or no duct at all. As used herein, a “duct” is a relatively simple tubular structure comprising glandular cells. A duct may be distinguished from a blood vessel by the presence of conspicuous cuboidal to columnar epithelial lining. There is only a single secretory unit (acinus or tubule) in a simple gland. Examples of simple glands are sweat glands, gastric glands, intestinal crypts, and uterine glands. A compound gland has a branching duct. Examples of compound glands include the salivary glands and the pancreas.

A gland may comprise an acinus. An “acinus” is a small ball of secretory epithelial cells containing a small central lumen. A compound acinar gland may comprise multiple ball-shaped acini interconnected by multiple ducts. Or, the secretory cells may be arranged as tubules. “Tubules” are elongated glands that have a lumen. Example tubular glands are sweat glands, gastric glands, and various mucous glands. Glandular cells may also comprise cords. “Cords” are epithelial cells arranged into sheets separated by vascular sinusoids. Generally, the pattern of cords is linear. Glands having cords include the pancreas (pancreatic islets), the parathyroid, adrenal cortex, and liver.

As used herein, a “biological material” may comprise a composition that includes at least one component that is derived from a biological source such as a cell, or an organ, or a part of a cell or an organ. A biological material may comprise a plurality of cells. Or, parts of cells may be accessed. As used herein, a cellular material may comprise cells and/or parts of cells. A part of a cell may comprise a subcellular organelle. Or a part of a cell may comprise a nucleic acid, such as DNA, messenger RNA or ribosomal RNA. Or, a part of a cell may comprise a protein. A biological material may comprise a composition that is secreted from an organ, such as seminal fluid or breast milk. Or, the biological material may be cells exfoliated or dislodged from the surface of a physiological structure such as an organ or a part thereof (e.g., a duct in a secretory organ). Natural body fluids such as serum, blood, breast milk, sputum, bronchial lavage fluid, eye fluid, urine and stool each are a biological material as used herein.

Also, as used herein, an “abnormal cell” or “transformed cell” is one that is different from a typical cell either on the basis of overall physiology, structure, or at the molecular level. For example, an abnormal (transformed) cell may appear different upon optical inspection. Or, an abnormal (transformed) cell may have altered staining of subcellular organelles. Or an abnormal (transformed) cell may comprise an alteration in the proteins produced by the cell. Or an abnormal (transformed) cell may have alterations in the DNA. The abnormal cell may be cancerous or precancerous. As used herein, a cancer cell is a cell that proliferates or grows in a uncontrolled manner. A cancer cell has the ability to form a malignant tumor and/or metastasize. Also, as used herein, a precancerous cell is a cell that is abnormal, and has a known propensity to develop into a cancer, but is not yet malignant.

As used herein, a “molecular characteristic correlated with cancer” may comprise any molecular change that may progress to cancer. The molecular change may be a change in the structure of the cell's DNA, RNA, or a protein. Or, the molecular change may be a change in gene expression, or protein synthesis. Examples of molecular characteristics that may be detected using the methods, devices and systems of the present invention comprise changes in cell morphology. Or, molecular characteristics that may be detected using the methods, devices and systems of the present invention comprise changes in protein synthesis or protein stability. Or, the molecular change may comprise a mutation of the DNA sequence, or a change in the structure of the DNA, such as a change in the methylation of the DNA, or instability of the DNA duplex or a chromosome. Or, the molecular change may comprise a change in gene expression.

As used herein, a “computer program” comprises a computer-encoded language that encodes the steps required for the computer to perform a specific task or tasks. Also, as used herein, “software” comprises the computer program(s) used in conjunction with any other operating systems required for computer function.

Also, as used herein, “computer-readable media” include, but are not limited to, an electronic, optical, magnetic, or other storage or transmission device capable of providing a processor with computer-readable instructions. Other examples of suitable media include, but are not limited to, a floppy disk, CD-ROM, magnetic disk, memory chip, ROM, RAM, an ASIC, a configured processor, all optical media, all magnetic tape or other magnetic media, or any other medium from which a computer processor can read instructions. Also, various other forms of computer-readable media may transmit or carry instructions to a computer, including a router, private or public network, or other transmission device or channel, both wired and wireless. The instructions may comprise code from any computer-programming language, including, for example, C, VISUAL C#®, VISUAL BASIC®, VISUAL FOXPRO®, Java, and JavaScript.

As used herein, a “computer processor” or “CPU” may include, for example, digital logic processors capable of processing input, executing algorithms, and generating output as necessary in response to the inputs received from an input device. Such processors may include a microprocessor, such as an ASIC, and state machines. Such processors include, or may be in communication with, media, for example computer-readable media, which stores instructions that, when executed by the processor, cause the processor to perform the steps described herein.

Embodiments of the present invention comprise methods, devices, and systems for detection of cancer. For example, embodiments of the present invention may comprise methods for testing a subject for the presence of cancer cells in an organ in a subject. In one embodiment, the method may comprise the steps of: (a) accessing a biological material by causing the biological material to be emitted from the organ; (b) collecting the biological material; and (c) examining the biological material for cells comprising at least one molecular characteristic correlated with cancer. In an embodiment, the biological material comprises a plurality of cells. Alternatively, the biological material may comprise a plurality of parts of cells. In an embodiment, the cells or parts of cells comprise a fluid suspension.

In an embodiment, the step of accessing a biological material may comprise causing a biological material to be secreted from the organ. Alternatively, the step of accessing a biological material may comprise causing a biological material to be dislodged from an organ. For example, in one embodiment, massage may be used to dislodge a biological material from an organ. Or, the organ may be flushed with a fluid to dislodge a biological material. Also, in an embodiment, the step of examining the biological material may comprise optical examination.

The subject may comprise a mammal. In an embodiment, the subject may comprise a human. The cancer may be specific to a particular organ or body part. In one embodiment, the organ may comprise a glandular organ. For example, the organ may comprise the prostate. The organ may also comprise a mammary gland. Or the organ may comprise a pancreas. In yet another embodiment, the organ may comprise a thyroid gland. The organ may also comprise an adrenal gland. In yet another embodiment, the organ may comprise a testis. Or, the organ may comprise an ovary. In another embodiment, the organ may comprise a liver. The organ may also comprise a saliva gland or a gall bladder.

In another embodiment, the cancer may be specific to an organ that does not necessarily comprise glandular tissue. For example, in one embodiment, the cancer may comprise epithelial cells exfoliated from the surface of an organ. The cells may be dislodged from the organ by flushing the organ with a liquid and collecting the liquid. In one embodiment, the cells may comprise cells exfoliated or dislodged from the digestive tract. For example, the colon and other portions of the intestines may be flushed with a biologically compatible liquid and cells that are exfoliated or otherwise dislodged from the surface of the intestines collected and examined using the methods, devices and systems of the present invention. The liquid used for such flushing may comprise a solution that allows for the majority of the liquid to pass through the colon, rather than the bladder, such as fluids currently used to prepare the colon for a standard colonoscopy. In an embodiment, the entire gastrointestinal system (e.g., mouth, salivary glands, esophagus, stomach, gall bladder, duodenum, pancreas, small intestine, and colon), or a portion thereof, may be flushed by drinking such a liquid, or flushing portions of the tract, with a liquid that may clear cells as the fluid is passed through the system. In yet another embodiment, a mouthwash may be used to collect saliva and cells from the mouth.

In an embodiment, mechanical means may be used to dislodge cells and allow them to be carried by the flow of the biological material secreted by an organ. In one embodiment, massage may be used. The massage may be manual or assisted by tools. In alternate embodiments, mechanical vibrations from few Hertz to Mega-Hertz (ultrasound) frequencies may be applied.

Or, the biological material may comprise a body fluid. Body fluids from several types of cancer have be successfully used for the detection of neoplasia (Sidransky, D., Science, 278, 1054-1059, 1997; Hoque, M. O., et al., Cancer Res., 63:5723-5726, 2003). Cells may be exfoliated from the kidney, prostate or the bladder and collected as a specimen of urine. For example, prostate cancer cells may be recovered from urine for further analysis (Hoque, M. O., et al., J. Clin. Oncol., 23:6569-6575, 2005). Also, other body fluids may be used. Stool may be collected for recovery of colon cancer or pancreatic cancer cells. In other embodiments, cells from liver (Wong, I. H., et al., Cancer Res., 59:71-73, 1999), breast (Silva, J. M., et al, Cancer Res., 59:3251-3256, 1999), or head and neck cancer (Sanchez-Cespedes, M. et al., Cancer Res., 60:892-895, 2000) may be recovered from serum. In another embodiment, cells dislodged from the surface of the lung may be recovered in serum (Esteller, M. et al., Cancer Res., 59:67-70, 1999) sputum (Belinsky S. A., et al., Proc. Natl. Acad. Sci., USA, 95:11891-11896, 1998) or bronchoalveolar lavage (Topaloglu, O., et al., Clin. Cancer Res., 10:2284-2288, 2004). Or, the cancer may comprise cells dislodged from the uterus (e.g., the cervical cancer cells). Other types of cancer cells may be detected using the methods and systems of the invention utilizing tissue collection methods available in the art.

In an embodiment, the present invention may comprise collecting a biological material from the organ of interest. The biological material may comprise a suspension of the cells in a fluid. Or, the biological material may comprise parts of cells, such as subcellular organelles or cellular membranes. Or, macromolecules such as chromatin, DNA, RNA or proteins may be accessed.

For example, where the organ is a prostate, prostate cells may be suspended with sperm in seminal fluid. Or, the prostate cancer or bladder cancer cells may be detected in a subject's urine. Or, where the organ is a breast, breast cells may be suspended in breast milk. Or, as described above, cells from the gastrointestinal (GI) tract may be suspended in a liquid flushing solution. Also, cells in the mouth may be suspended in a mouthwash solution. Or, the cells may be suspended in plasma, sputum, or another type of biological fluid.

The method may further comprise a step of at least partly separating a portion of the cells, or parts of cells (i.e., cellular material) from at least one other component of the biological material prior to the step of examining the biological material. For example, where the biological material is a suspension of a relatively small number of cells in a biological fluid, it may be necessary to concentrate the cells prior to examination. Thus, in one embodiment, the step of at least partly separating the cellular material from at least one other component of the biological material may comprise subjecting the biological material to a centrifugal force. Or the biological material may be subjected to filtration through a filter of defined pore size to concentrate the cells and/or separate out cells and macromolecules from smaller diluents such as water.

The method of examining the biological material may depend upon the type of biological material being analyzed. In one embodiment, cellular material in the biological material may be examined using a viewing device. The viewing device selected for use with the methods of the present invention may depend on the biological material being examined. In one embodiment, the viewing device may comprise a standard compound (or simple) microscope. Additionally or alternatively, the viewing device may comprise a standard film camera, a digital camera, or a movie camera. Both a still camera and a movie camera may use film or a digital detector such as a charge coupled device (CCD) or a complementary metal oxide semiconductor (CMOS) detector. A film may be scanned after development by a human or, for a digital film, by a computer. In an embodiment, a still digital camera or a movie camera may be connected to a computer for real-time review of the images. Alternatively, the camera output may go to a storage device for later review.

Also, optical enhancement devices such as filters, or spatial filtering may be used. In spatial filtering, the size of a cell, or a particular component of a cell (e.g., a nucleus) may trigger a filter step. For example, cells that comprise a nucleus that is abnormally large will pass the spatial filter and be projected as a bright image, whereas a normal sized nucleus will not be imaged. Such filtering can facilitate detection of abnormal cells by either a human or a computer program.

In one embodiment, the step of examining the biological material for cellular material comprising at least one molecular characteristic correlated with cancer may comprise viewing the cells with a microscope. For example, the cells may comprise a change in shape as they progress from a normal phenotype to a pre-cancerous or cancerous phenotype. Or, the cells may comprise a change in internal structure or biochemical composition that may be detected visually upon staining for the structure or compound of interest. For example, the abnormal cells may comprise a change in the amount of a particular protein that can be detected by staining or immunhistochemistry.

In one embodiment, the cells or parts of cells may be stationary on a microscope slide. Or, the biological material may comprise cells suspended in a liquid, such that the cells may be examined as the biological material moves past the lens of a microscope. Where the biological material comprises a liquid suspension of cells, the cells may be concentrated from the bulk of the fluid prior to, or during, the step of examination. Thus, the biological material may be subjected to a centrifugal force, and the portion of the material that comprises the cells of interest isolated for examination. Or, for real-time separation during the examination step, the method may comprise the step of causing the biological material to rotate in a chamber and examining at least a portion of the cells that are forced to the outer perimeter of the chamber. For example, the cells may be examined by causing the liquid to rotate in a chamber under a microscope objective, and inspecting the cells with the microscope trained at the side of the chamber, where one of the chamber walls is used as a window for the microscope.

In an embodiment, the biological material may be treated to enable detection of changes that may be diagnostic of cancer or to facilitate the detection and/or isolation of a cell population of interest. Thus, in one embodiment, the method may comprise exposing the biological material to an agent that can specifically interact with a marker on the surface of a specific cell type (e.g., prostate or breast cell). Or, the method may comprise exposing the biological material to an agent that can specifically interact with a marker on the surface of a malignant or precancerous cell. In one embodiment, the marker may comprise a protein and the agent that recognizes the marker may comprise an antibody that recognizes, and binds to, the protein. Or, the marker may comprise a receptor and the agent may comprise a ligand for the receptor. In yet another embodiment, the marker may comprise a fluorescent agent that can specifically bind to the cell type of interest.

The analysis of the biological material may, in certain embodiments, comprise the use of a computer. The computer program (i.e., programming code) may comprise a plurality of steps whereby a visual image of each of a plurality of cells are compared to an image representing other components of the biological material. For example, prostate cells in seminal fluid may be distinguished from sperm or non-prostate cells that may be present.

Alternatively or additionally, the computer program (i.e., programming code) may comprise a plurality of steps whereby a visual image of each of a plurality of cells are compared to an untransformed cell and an abnormal (i.e., precancerous or cancer) cell. If the image of the cell in the biological material comprises a predetermined similarity with the transformed cell image, there may be a selection step whereby the image is classified as a transformed (e.g., precancerous of cancer cell). If the image comprises a significant overlap with a normal image, the image may be classified as normal or untransformed tissue. The image may be saved for further review by a human. In an embodiment, there may be a pre-determined number of images that are registered as abnormal or transformed for the patient sample to be considered abnormal such that a follow-up examination is required.

In another embodiment, the present invention may comprise a device for examining a biological material for the presence of cells comprising at least one molecular characteristic correlated with cancer. The device may comprise a chamber having at least one portion that comprises a focal plane suitable for optical examination of the biological material using a viewing device.

In one embodiment, the biological material may be localized on a substrate that is stationary with respect to a viewing device. Or, the biological material may comprise a cellular material suspended in a liquid, and the cells may be examined as the biological material moves past the viewing device.

The viewing device selected for use with the devices of the present invention may depend on the biological material being examined. In one embodiment, the viewing device may comprise a standard compound (or simple) microscope. Additionally or alternatively, the viewing device may comprise a standard film camera, a digital camera, or a movie camera. Both a still camera and a movie camera may use film or a digital detector such as a CCD or a CMOS detector. A film may be scanned after development by a human or, for a digital film, by a computer. In an embodiment, a still digital camera or a movie camera may be connected to a computer for real-time review of the images. Alternatively, the camera output may go to a storage device for later review.

Also, optical enhancement devices such as filters, or spatial filtering may be used in certain embodiments of the devices of the present invention. In spatial filtering, the size of a cell, or a particular component of a cell (e.g., a nucleus) may trigger a filtering step. For example, cells that comprise a nucleus that is abnormally large will be recognized by the spatial filter and thereby projected as a bright image, whereas normal sized nucleus will not be imaged. Such filtering can facilitate detection of abnormal cells by either a human or a computer program.

To facilitate flow of a liquid biological material past the viewing device, the examination device chamber may, in certain embodiments, comprise a first reservoir for storing a first portion of the biological material prior to optical examination, and a channel for positioning a second portion of the biological material in the focal plane of a viewing device. The channel may comprise a first end and a second end such that while the cells or parts of cells are being examined, the biological material flows from the first end of the channel to the second end of the channel in a manner such that at least a portion of the flow comprises the focal plane of the viewing device. In one embodiment, the first reservoir for storing the first portion of the biological material is positioned on the first end of the channel such that the biological material moves from the first reservoir into the channel.

After the biological material flows past the viewing device, it may be collected or discarded. Thus, in an embodiment, the examination device may comprise a second reservoir for collecting the biological material after examination of the biological material by the viewing device. In an embodiment, the second reservoir is positioned on the second end of the channel such that the biological material moves from the channel to the second reservoir. Thus, the examination chamber may comprise a first reservoir for storing the biological material prior to viewing, and a second reservoir for receiving the biological material after the cells (or parts of cells) have traveled past the viewing device. In one embodiment, the biological material may flow from the first reservoir, through the channel, and into a second reservoir. Or, the biological material may flow from the first reservoir, through the channel, and then be discarded. Or, the biological material may flow from the first reservoir, through the channel, and into a second reservoir, and then be discarded.

Where the biological material comprises a liquid, the examination device may, in certain embodiments, comprise a means to concentrate the cellular material. In one embodiment, the device may rotate the biological material in a circular chamber such that at least a portion of the cells (or parts of cells) are forced to the perimeter of the chamber for examination by the viewing device. Thus, in one embodiment, the chamber for positioning the cells in the focal plane of the viewing device may comprise a circular chamber having a rotating device, such as a plurality of rotating blades, such that the cells are forced to the wall of the chamber.

In an embodiment, where the biological material comprises cells suspended in a fluid, the flow of the biological material may be parallel to the focal plane of the viewing device. For example, where the size of the cells, or the geometry of the device, is such that a substantial portion of the cells are positioned in the focal plane as the cells move past the viewing device, then the device may comprise a focal plane that is parallel to the flow of the biological material to maximize the viewing time for each cell. In some cases, where the fluid will not easily flow through a narrow gap in a time that allows for proper or efficient examination of the biological material, a larger flow volume may be used for optical examination. The larger volume may allow for some cellular material to pass either further away from the microscope objective, or nearer to the microscope objective, such that a portion of the cells are out of a focal plane that is parallel to the flow of the biological material. Where cells pass outside of the focal plane, it may be difficult to obtain an image suitable for examination and analysis. In this case, it may be preferred to have the focal plane be angled with respect to the flow of biological material past the viewing device. An angled focal plane may provide for the viewing of cellular material that is positioned at various depths as they flow past the viewing device.

In yet another embodiment, the present invention may comprise a system for examining a biological material for the presence of at least one molecular characteristic correlated with cancer. In an embodiment, the biological material comprises a plurality of cells. Alternatively, the biological material may comprise a plurality of parts of cells. In an embodiment, the cells or parts of cells comprise a fluid suspension.

In one embodiment, the cells or parts of cells may be localized on a substrate that is stationary with respect to a viewing device. Or, the biological material may comprise cells or parts of cells suspended in a liquid, and the cellular material may be examined as the biological material moves past the viewing device.

The viewing device selected for use with the devices of the present invention may depend on the biological material being examined. In one embodiment, the viewing device may comprise a standard compound (or simple) microscope. Additionally or alternatively, the viewing device may comprise a standard film camera, a digital camera, or a movie camera. Both a still camera and a movie camera may use film or a digital detector such as a CCD or a CMOS detector. A film may be scanned after development by a human or, for a digital film, by a computer. In an embodiment, a still digital camera or a movie camera may be connected to a computer for real-time review of the images. Alternatively, the camera output may go to a storage device for later review. Also, optical enhancement devices such as filters, or spatial filtering may be used to facilitate detection of abnormal cells by either a human or a computer program.

To facilitate flow of a liquid biological material past the viewing device of the system, the examination device chamber may comprise a first reservoir for storing a first portion of the biological material prior to optical examination, and a channel for positioning a second portion of the biological material in the focal plane of a viewing device. The channel may comprise a first end and a second end such that while the cells or parts of cells are being examined, the biological material flows from the first end of the channel to the second end of the channel in a manner such that at least a portion of the flow comprises the focal plane of the viewing device. In one embodiment, the first reservoir for storing the first portion of the biological material is positioned on the first end of the channel such that the biological material moves from the first reservoir into the channel.

After the biological material flows past the viewing device, it may be collected or discarded. Thus, in certain embodiments, the examination device of the system may comprise a second reservoir for collecting the biological material after examination of the biological material by the viewing device. In an embodiment, the second reservoir is positioned on the second end of the channel such that the biological material moves from the channel to the second reservoir. Thus, the examination chamber may comprise a first reservoir for storing the biological material prior to viewing, and a second reservoir for receiving the biological material after the biological material has traveled past the viewing device. In one embodiment, the biological material may flow from the first reservoir, through the channel, and into a second reservoir. Or, the biological material may flow from the first reservoir, through the channel, and then be discarded. Or, the biological material may flow from the first reservoir, through the channel, and into a second reservoir, and then be discarded.

The system may further comprise a means to cause a liquid biological material to flow past the viewing device for examination. Gravity may be utilized as a means to cause the flow of the biological material past the objective of the viewing device. In one embodiment, gravity is used to cause the flow of a liquid suspension of cells or parts of cells from a first reservoir, through the channel and into a second reservoir. Additionally or alternatively, the system may comprise a flow inducer to apply positive pressure to the first chamber to thereby push the biological material into the focal plane of the second chamber.

Where the biological material comprises a liquid, the examination device may, in some embodiments, comprise a means to concentrate the cellular material. In one embodiment, the device may rotate the biological material in a circular chamber such that at least a portion of the cells or parts of cells are forced to the perimeter of the chamber for examination by the viewing device. Thus, in one embodiment, the chamber for positioning the cells in the focal plane of the viewing device may comprise a circular chamber having a rotating device, such as a plurality of rotating blades, such that the cells are forced to the wall of the chamber.

In one embodiment, the flow of the biological material may be parallel to the focal plane of the viewing device. In other embodiments, however, it may be preferred to have larger dimensions for the biological material to flow past the viewing device. This can allow for the analysis of greater numbers of cells or prevent clogging of the examination device with cells in the biological material. The larger flow volume may allow for some cells or cell parts to pass further away from the microscope objective, or nearer to the objective, such that a portion of the cells or cell parts are outside of a focal plane that is parallel to the flow of the biological material. Where the dimensions of the flow of the biological material allow for a plurality of cells to pass the viewing device at one time, the focal plane of the viewing device may be angled with respect to the flow of cells through the second part of the chamber and past the viewing device. An angled focal plan may provide for the viewing of cells that are positioned at various depths as they flow past the viewing device.

In an embodiment, the system may further comprise a means to analyze images obtained upon examination of the biological material of interest. In an embodiment, the system may comprise a means to collect the visual images obtained upon inspection of the biological material of interest. The means to collect the visual images may comprise a digital camera. Alternatively or additionally, the images may be enhanced using digital processing.

The analysis of the biological material by the system may, in certain embodiments, comprise the use of a computer. The computer program (i.e., programming code) may comprise a plurality of steps whereby a visual image of each of a plurality of cells are compared to an image representing other components of the biological material. For example, prostate cells in seminal fluid may be distinguished from sperm or non-prostate cells that may be present.

Alternatively or additionally, the computer program (i.e., programming code) of the system may comprise a plurality of steps whereby a visual image of each of a plurality of cells are compared to an untransformed cell and an abnormal (i.e., precancerous or cancer) cell. If the image of the cell in the biological material comprises a predetermined similarity with the transformed cell image, there may be a selection step whereby the image is classified as a transformed (e.g., precancerous of cancer cell). If the image comprises a significant overlap with a normal image, the image may be classified as normal or untransformed tissue. In an embodiment, the image may be saved for further review by a human. In an embodiment, there may be a pre-determined number of images that are registered as abnormal or transformed for the patient sample to be considered abnormal such that a follow-up examination is required.

In an embodiment, the present invention further comprises computer-readable media which may allow the methods, devices, and systems of the present invention to be automated or used by a plurality of users. Thus, in an embodiment, the invention comprises a computer-readable medium on which is encoded programming code for the analysis of a biological material for the presence of cells comprising at least one molecular characteristic correlated with cancer. The computer program (i.e., programming code) may comprise a plurality of steps whereby a visual image of each of a plurality of cells are compared to an image representing other components of the biological material. For example, prostate cells in seminal fluid may be distinguished from sperm or non-prostate cells that may be present. Alternatively or additionally, the computer program (i.e., programming code) may comprise a series of steps whereby a visual image of each of a plurality of cells are compared to an image representing an untransformed cell and an abnormal (i.e., precancerous or cancer) cell. If the image of the cell in the biological material comprises a predetermined similarity with the transformed cell image, there may be a selection step whereby the image is classified as a transformed (e.g., precancerous or cancer cell). If the image comprises a significant overlap with a normal image, the image may be classified as normal or untransformed tissue. In an embodiment, the image may be saved for further review. In an embodiment, there may be a pre-determined number of images that are registered as abnormal or transformed for the patient sample to be considered abnormal such that a follow-up examination is required.

Thus, the present invention relates to early cancer detection. The detected cancer may be cells that are secreted or dislodged from an organ. Cancers of glandular organs may secrete cancerous cells or parts of cells into the effluent of the gland. For example, prostate cancer cells may be secreted into the seminal fluid. Similarly, breast cancer cells may be secreted into milk produced by a lactating breast. Also, many common human cancers arise from the epithelial surface of an organ. Such cancers may include colon cancer, lung cancer, prostate cancer, uterine cancer, bladder cancer and cervical cancer. Development of such cancers may result in the exfoliation of cancerous cells into various bodily fluids or effluents such as sputum, bronchial lavage fluid, seminal fluid, stool, or urine. Or, the organ may be flushed with a biologically compatible liquid as a means to dislodge cells from the surface of the organ.

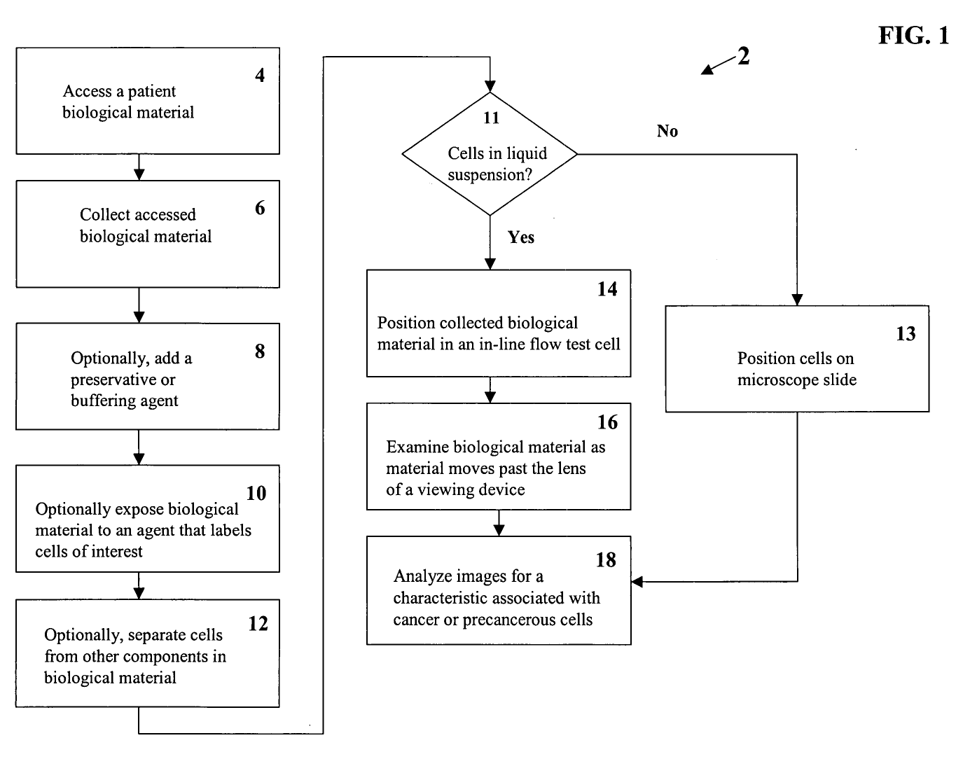

FIG. 1 shows a flow-diagram of an embodiment of a method 2 of the present invention for detection of cancer cells from a gland or from the surface of an organ. Thus, as shown in FIG. 1, the method may comprise a first step 4 of accessing a biological material by causing the biological material to be emitted from an organ. In an embodiment, the biological material comprises a plurality of cells from an organ of interest. Or, the biological material may comprise parts of cells, such as a subcellular organelle or macromolecule. The step of accessing the biological material may comprise inducing a gland of interest to secrete a biological material or causing cells or cell parts to be dislodged from the surface of an organ. Or, the step of accessing the biological material may comprise collecting a bodily fluid that may comprise the cells or cell parts of interest. The gland may comprise breast tissue, or prostate tissue or other secretory glands. Or, the organ may comprise the bladder, lung, or the gastrointestinal (G.I.) tract (e.g., mouth, esophagus, stomach, or intestines).

In an embodiment, the present invention provides for early detection of a cancer of a glandular organ by causing the organ to secrete a biological material that is normally produced by the gland as a means to collect cells that are derived from a tumor or a precancerous growth. Thus, one embodiment of the invention may comprise a method of testing for malignancy of a gland in a subject by causing the gland to secrete a biological material, collecting the secreted biological material, and examining the cells in the secreted materials.

For example, the gland may be human breast. The mammary gland is a milk-producing structure that is composed largely of fat cells. Within the mammary gland there is a complex network of branching ducts that emanate from sac-like lobules. The lobules are the glands that produce milk, and the ducts transport milk from the lobules to the nipple. Thus, in the case of a breast, a woman may be given a dose of a lactogen to cause the breasts to generate milk. The lactogen may comprise a lactogenic hormone. In alternate embodiments, the lactogen may comprise a Prolactin or Luteotropin. Or, other compounds known to induce production of breast milk may be used. For the analysis of cells in the breast milk, the milk secreted by the breast lobules into the ducts may be collected via the nipple. Cells that line the inside of the globules or the ducts may be carried with the milk to the nipple, where the liquid can be colleted for testing.

Or, the organ may comprise a prostate. In the case of the prostate, a man may be made to ejaculate and seminal fluid comprising prostate cells colleted for testing. Or, prostate cells may be collected from urine. For example, a sample of urine (e.g., 50-100 milliliters (ml)) may be collected and cells in the urine pelleted by centrifugation at 3,000×g for 10 minutes (Hoque, M. O., et al, J. Clin, Oncol., 23:6569-6675, 2005). The cells may be washed with a physiological buffer solution (e.g., phosphate buffered saline) and the cells stored or processed for further analysis.

Or cells may be collected from the sputum that emanates from the lung. In this case, a subject may be induced to cough and the sputum collected (Belinsky S. A., et al., Proc. Natl. Acad. Sci., USA, 95:11891-11896, 1998). Or, cells dislodged from the surface of the lung may be recovered in serum (Esteller, M. et al., Cancer Res., 59:67-70, 1999) or bronchoalveolar lavage (Topaloglu, O., et al., Clin. Cancer Res., 10:2284-2288, 2004).

In an embodiment, mechanical means may be used to dislodge cells and allow them to be carried by the flow of the biological material secreted by an organ. In one embodiment, massage may be used. The massage may be manual or assisted by tools. Mechanical vibrations from few Hertz to Mega-Hertz (ultrasound) frequencies may be applied. Or, in some cases the surface of the organ may be scraped to dislodge cells.

In some cases, the tumor may not be located on the inside surface of a globule or duct. Still, the cancer may be detected by inducing such cells to be secreted. For example, by massaging the prostate, cells present at the site of a tumor may be dislodged and secreted. Or carcerous cells that are not directly at the inside surface of a globule or a duct may be detected after the cancer develops and penetrates into the inside of a duct or a globule. In many cases, this may still be an early stage where the tumor is sub-millimeter in size.

Alternatively, the cancer may be specific to a biological material that does not necessarily comprise glandular secretion. The cancer may comprise epithelial cells exfoliated from the surface of an organ. For example, cells may be exfoliated from the kidney, prostate or the bladder and collected as a specimen of urine. Also, stool may be collected for recovery of colon cancer or pancreatic cancer cells. In other embodiments, cells from liver (Wong, I. H., et al., Cancer Res., 59:71-73, 1999), breast (Silva, J. M., et al, Cancer Res., 59:3251-3256, 1999), or head and neck (Sanchez-Cespedes, M. et al., Cancer Res., 60:892-895, 2000) cancer may be recovered from serum. Or, the cancer may comprise cells dislodged from the uterus (e.g., the cervical cancer cells).

Or, the cells may be dislodged from the organ by flushing the organ with a liquid and collecting the liquid. For example, the colon and other portions of the intestines may be flushed with a biologically compatible liquid and cells that are exfoliated or otherwise dislodged from the surface of the intestines collected. Or, the gastrointestinal system, or portions thereof, may be flushed by drinking a liquid that may clear cells once the fluid is passed through the system. In yet another embodiment, a mouthwash may be used to collect saliva and/or cells from the mouth.

Next, the accessed biological material may be collected 6 (FIG. 1). For example, milk secreted from a breast may be collected by pumping the milk out of the nipple. Or, for the analysis of prostate cells, seminal fluid or urine may be collected. Or, the liquid used to flush portions of the gastrointestinal tract may be collected.

In some cases, external agents may be added to the collected biological material 8 (FIG. 1). For example, a preservative, a buffering agent, or an agent to prevent coagulation may be added to the collected biological material. Such chemicals may include, but are not limited to, a physiological buffer (e.g., Tris buffer, citrate buffer, phosphate buffer) ethylenediamine tetraacetic acid (EDTA), a salt or a sugar to adjust the osmolarity, an anti-oxidant, or other preservatives.

The biological material may be treated to enhance the imaging of abnormal (e.g., pre-cancerous or cancerous) cells. Thus, the method may include the step whereby the biological material may be exposed to an agent that may tag (i.e., label) the cellular material of interest 10 (FIG. 1).

For example, to label prostate cells, the biological material may be exposed to an antibody that recognizes a cell-specific protein. For example, for detection of prostate cells, the biological material may be exposed to an antibody to prostate-specific membrane antigen (PSMA). Even in the case where a marker such as a cell-surface antigen is not specific to a cancer cell, such as PMSA, the marker may allow for the cells that are derived from the organ of interest (e.g., prostate cells) to be distinguished from other types of cells (e.g., sperm cells, lymphocytes or other serum cells). In this way, the cells of interest may be preferentially selected from a background of other cell types.

Or, the biological material may be exposed to an antibody that can bind to a protein expressed on the surface of a cancer cell. For example, the epidermal growth factor receptor (EGFR) has been reported to be expressed in high levels in primary breast cancer (Leitzel et al., 1998, Clin. Cancer Res., 4:3037-3043). Thus, for the detection of breast cancer cells, a ligand that recognizes EGFR may be used.

In an embodiment, the agent that preferentially binds to the cell type of interest may be used to identify and/or purify cells of interest. For example, an antibody to a cell-specific or a cancer-specific protein may be labeled with gold nanoparticles such that cells that bind the antibody can be visualized with a light microscope. Or, the antibody may be labeled with magnetic beads such that cells that bind the antibody can be isolated by immunomagnetic sorting as is known in the art. Or, the antibody may be labeled with a fluorescent molecule (e.g., fluorescein) for cell sorting by optical detection. Such fluorescently labeled cells may be selected by altered deflection of droplets comprising the labeled cells as compared to unlabeled cells by compressed air. Or, the biological material may be incubated with a first antibody that recognizes the cells of interest, and a second labeled antibody that binds to the first antibody, where the second antibody is labeled with a detectable molecule such as a fluorophore or an enzyme.

The cellular material may be separated from other components in the biological material 12 (FIG. 1). In some cases, it may be necessary to concentrate the cellular material. If the biological material obtained has relatively few cells in a large volume of liquid, several processes may be used to concentrate the cells and reduce the volume of the liquid before examining the cellular material. The biological material may be subjected to a centrifugal force. In this way, materials in the liquid are segregated according to specific gravity. For example, the biological material may be subjected to a centrifugal force to separate prostate cells from semen and the seminal fluid. Or the biological material may be subjected to filtration through a filter of defined pore size to concentrate and/or separate out cells and macromolecules from smaller diluents such as water.

The centrifugation conditions may be chosen such that the cells of interest may create a distinct layer in the centrifuge tube and can be extracted for examination. In alternate embodiments, the biological material may be centrifuged at about 500×g to about 20,000×g, or from about 1,000×g to about 15,000×g, or from about 2,000×g to about 7,500×g. Depending upon the centrifugal force applied, the biological material may be centrifuged for a time in the range of from about 30 seconds to about 45 minutes, or from about 2 minutes to about 20 minutes or about 3 minutes to about 10 minutes. In one example, the biological material may be centrifuged at 3,000×g for about 10 minutes and the cells collected. The cells may then be saved or processed further. For example, in an embodiment, the cells may be reconstituted in a reduced volume of a physiological solution such as phosphate buffered saline (PBS).

The biological material may then be positioned in a test chamber to allow for the examination of the cells of interest. There may be a decision 11 as to the type of examination device employed. In one embodiment, the cells in the biological material may be sufficiently concentrated so they can be spread on a microscope slide 13 and examined for abnormal shape, staining or structure. Alternatively, in some cases the biological material may not have been concentrated. Or, the cells may have been at least partly separated from other components in the biological material, but may still comprise a suspension (or resuspension) of cells in a fluid. Such liquid biological materials may be positioned in an in-line flow cell 14 (FIG. 1)

For the biological material that is examined using an in-line flow cell, the cellular material of interest may then be caused to move past the lens of a microscope or other viewing device and examined for abnormal shape, staining or structure 16. For example, pressure may be applied to the biological material to cause the cellular material to flow through a channel that allows for microscopic examination of the cells of interest such that the cells may be identified as cancer or precancerous in phenotype.

The analysis may comprise the determination of whether any of the cells or cell parts comprise at least one molecular characteristic correlated with cancer 18. The type of analysis may vary depending upon the biological material being analyzed. The molecular characteristic correlated with cancer may be a change in the structure of the cell's RNA or a protein. Or, the molecular change may comprise a mutation in the DNA, or a change in the structure of the DNA, such as changes in the methylation of the DNA, or instability of the DNA duplex. The molecular change may also comprise a change in gene expression. Or, the molecular change may comprise a change in the expression of a particular protein. Examples of molecular characteristics that may be detected using the methods, devices and systems of the present invention comprise changes that lead to a change in cell morphology. In one embodiment, images of cells in the biological material may be analyzed for a characteristic associated with cancer or precancerous cells.

Thus, the biological material may be examined by separating the cells from liquids and other materials in a centrifuge, spreading the cells on a microscope slide, and inspecting the cells under a microscope. Or, an in-line flow microscope may be used for examination of the biological material. For example, in an embodiment, the cells may be examined by causing a liquid biological material to flow in a narrow channel under a microscope objective, and inspecting the cells or cell parts with the microscope. An in-line flow microscope may comprise an in-line flow cell, where the liquid secreted from the gland including the cells is made to flow in a narrow gap under a microscope's objective.

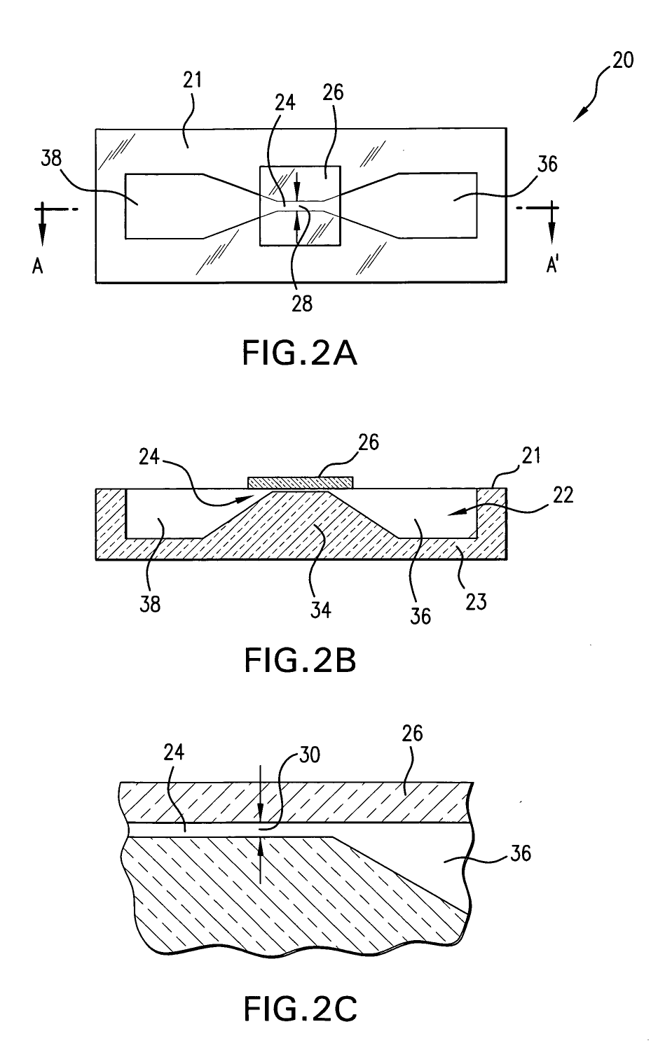

An in-line flow test cell that may be used with the methods and systems of the present invention is shown in FIG. 2. FIG. 2A shows a top view of a test cell 20 of the present invention, FIG. 2B shows a cross-section at the plane defined by A-A′, and FIG. 2C shows an enlarged view of the channel and cover glass. The test cell may comprise an upper surface 21 and a lower surface 23 (FIG. 2B). The test cell may include walls 34 to form a chamber 22 for placing a liquid biological material. The chamber may include a shallow and narrow recess 24 for the liquid to flow through while the cell is positioned to be viewed by a microscope. The recess 24 may become a channel after a microscope cover glass 26 is placed over the central part of the test cell. Thus, the chamber 22 may comprise a first reservoir 36 and a second reservoir 38 positioned on either side of the channel 24. As described below, the biological material may be caused to flow from the first reservoir 36, through the channel 24, and into second reservoir 38.

In another embodiment, there may not be a cover glass 26, but upper surface 21 may comprise a region above the channel 24 that is thin enough to allow optical viewing of the biological material in the channel. Thus, in one embodiment, upper surface 21 may comprise a single uninterrupted surface.

The width 28 of the channel 24 may be made to match the width of the field-of-view of a microscope or other viewing device. In alternate embodiments, the width of the channel may range from about 5 micrometers (μm) to about 10 mm, or from about 20 μm to about 1 mm, or from about 100 μm to about 600 μm. The depth 30 of the channel 24 may be such that the flow of liquid is limited to a single cell or can accommodate a few cells. In alternate embodiments, the depth of the channel may range from about 20 nanometers (nm) to about 1000 μm, or from about 1 μm to about 200 μm, or from about 10 μm to about 100 μm.

The in-line cell (i.e., test cell) may comprise dimensions as required for analysis of the biological material of interest. For example, the in-line flow cell may be sized to contain a single volume of reconstituted biological material as the analyte. In alternate embodiments, for smaller volumes, the first and second reservoirs may comprise a volume in the range of from about 20 microliters (μl) to about 20 ml, or from about 50 μl to about 5 ml, or from about 100 μl to about 2 ml. Thus, the device may comprise a length of about 2 cm to about 20 cm, and a height and width of from about 0.5 cm to about 6 cm, or a length of about 3 cm to about 15 cm, and a height and width of from about 1 cm to about 4 cm, or a length of about 5 cm to about 10 cm, and a height and width of from about 2 cm to about 3 cm.

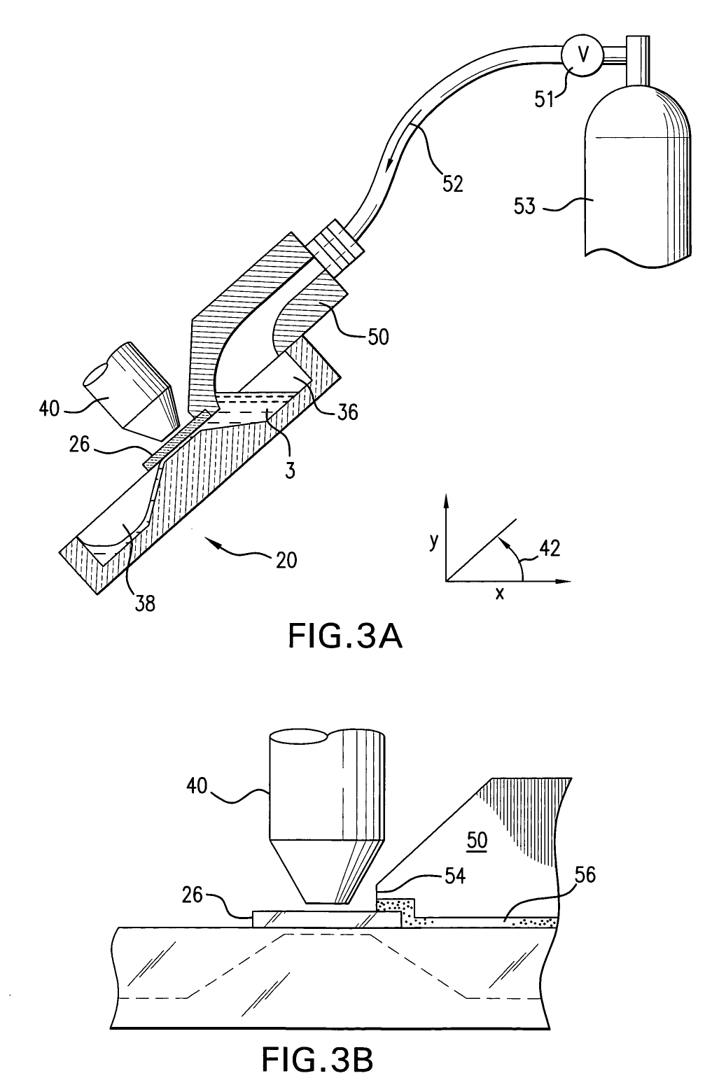

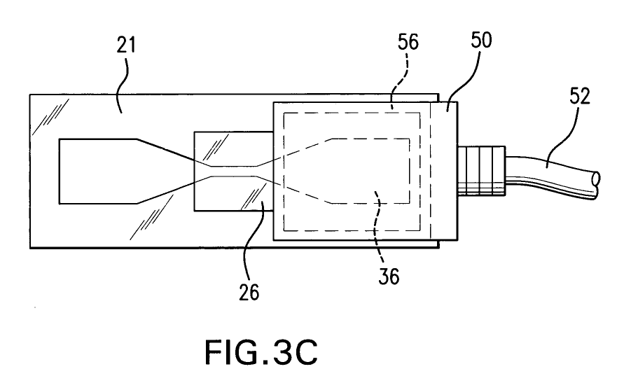

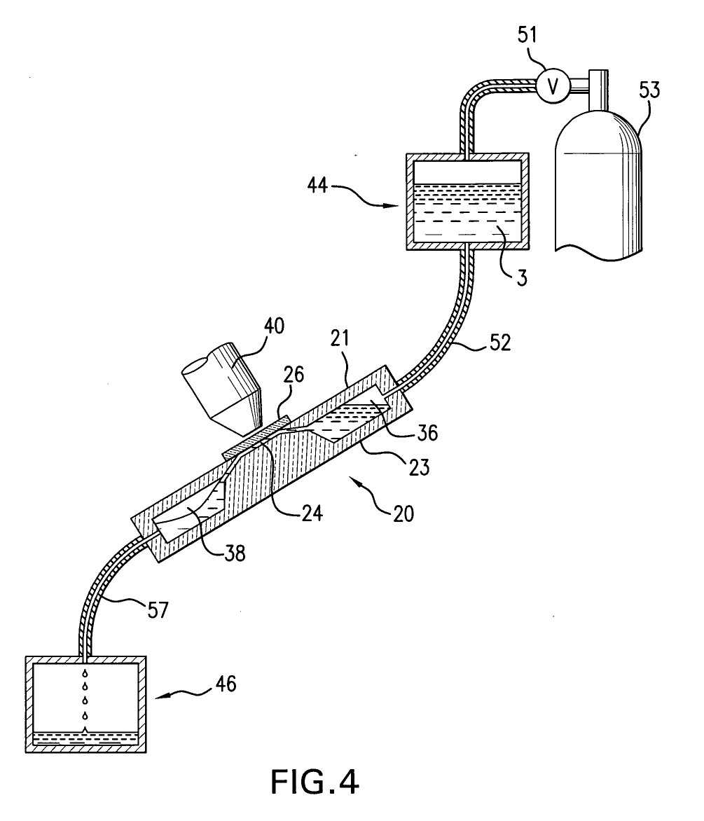

For larger volumes of the biological material (e.g., such as for examination of serum or a flushing solution), the device may comprise inlets and outlets to provide a flow-through apparatus as described below (FIGS. 3 and 4). Additionally or alternatively, correspondingly larger dimensions for the device 20 may be used. For example, for a larger volume of biological material, the first and second reservoirs may comprise a volume in the range of from about 2 ml to about 500 ml, or from about 5 ml to about 100 ml, or from about 10 ml to about 20 ml. Thus, the device may comprise a length of about 8 cm to about 50 cm, and a height and width of from about 5 cm to about 30 cm, or a length of about 12 cm to about 40 cm, and a height and width of from about 10 cm to about 20 cm, or a length of about 10 cm to about 20 cm, and a height and width of from about 10 cm to about 15 cm.

The test cell 2 may be made of materials that are suitable for containing and viewing the biological material. The cover glass 26 should be transparent to the light being used to view the cells. Also, the cover glass 26 may be flat, with parallel surfaces. In alternate embodiments, the cover glass may comprise a thickness that ranges from 0.01 mm to about 1 mm, or from about 0.05 mm to about 0.5 mm, or from about 0.1 mm to about 0.2 mm. In one embodiment, a standard microscope coverglass that is about 0.1 mm thick and 1.5-2 cm by 1.5-2 cm may be used.

Also, in an embodiment, the device may allow for the sample in the focal plane of the viewing device to be illuminated from below. For example, the chamber walls 34 may be made of glass or quartz. Or, the chamber walls may be made of a plastic, such as polystyrene, polycarbonate, polyethylene, polypropylene, or the like. Or the chamber walls may be made of a mineral or a ceramic such as fused quartz. Or, when top illumination is used, the chamber walls may be made of a nontransparent material. In one embodiment a metal, such as stainless steel, may be used.

Using the in-line flow device, the width and depth of the channel should be such that cells and other materials in the liquid can pass without clogging the channel, but not so wide such that a large proportion of the cells may be out of the field of view, or out of focus for the microscope. Alternatively, the channel may be made larger then the depth of focus of the microscope by having the microscope field-of-view angled to cover the depth of the channel, such that the cells are in focus at some point along their travel in the channel.

FIG. 3A shows a configuration for a test system of the present invention. Since the channel 24 is restricted in size, the liquid biological material 3 may be caused to flow through the channel by some means. As shown in FIG. 3, the in-line flow cell 20 may be placed at an angle 42 to allow the liquid 3 to flow from the first reservoir 36, through the channel 24, and into the second reservoir 38. A microscope objective 40 may be trained on the channel to view the flow of liquid and included cells.

Additionally or alternatively, a flow inducer 50 may be used to seal at least the first reservoir 36 and to introduce pressure into the first reservoir (FIG. 3A). As further illustrated in FIGS. 3B and 3C, the flow inducer may be further designed to have a lip 54 or other means that may function to hold the cover glass 26 in place. Also, the flow inducer may comprise a gasket 56 to seal the junction between the inducer and the test cell (FIGS. 3B and 3C). Using a flow inducer to apply positive pressure to the biological material and thereby cause the biological material to flow through the channel may negate the need to angle the in-line flow cell.

In an embodiment, a pressurized gas (e.g., air or nitrogen) may be used to force the biological material 3 from a first reservoir 36 through the channel 24 and into the second reservoir 38. Or a liquid may be used to force the biological material 3 from the first reservoir 36 into the channel 24. For example, where the biological material is primarily an aqueous liquid, an oil may be used to force the aqueous fluid through the channel 24. Or, where the biological material comprises a large number cells in a relatively small volume of liquid so that the suspension is highly viscous (e.g., a cell preparation concentrated by centrifugation or other means), the liquid may comprise an aqueous solution, such as an isotonic buffer. In one embodiment, the pressure may be controlled to create the desired flow speed. For example, the pressure may be controlled by regulating the pressure of the gas introduced via a tank 53 connected via a valve 51 and connector tubing 52.

After the biological material flows through the channel 24 it may be collected in the second reservoir 38 (FIG. 3A). The chamber may be sized such that once the biological material flows from the first reservoir 36 of the chamber to the second reservoir 38, the spent biological material may be collected in the second reservoir. The collected material may be discarded or stored for further use.

As shown in FIG. 4, in an alternate embodiment, the system may comprise a flow-through device. In an embodiment, the flow-through device may comprise a configuration such that material is pumped from a sample container 44 via connector tubing 52 into the first reservoir 36, through the channel 24, and into the second reservoir 38, and out via a connector tubing 57 and to a waste bin 46. In the flow-through device, the channel 24, and individual reservoirs 36,38 comprising the chamber 22 may be closed on the upper surface 21. In an embodiment, the cover glass may be attached to the optical device by reversible clamping or an adhesive. In another embodiment, there may not be a cover glass, but upper surface 21 may comprise a region above the channel 24 that is thin enough to allow optical viewing of the biological material in the channel. Thus, in one embodiment, upper surface 21 may comprise a single uninterrupted surface.

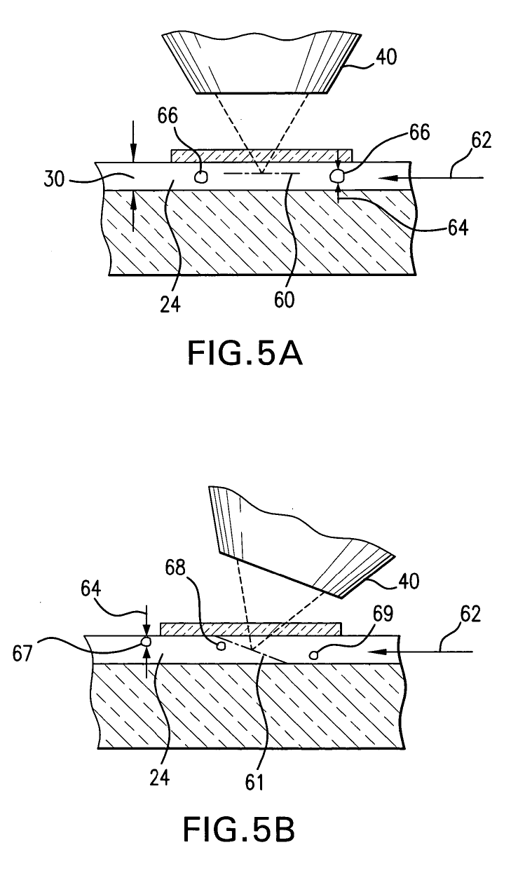

The viewing device may be positioned to allow for the focal plane to be optimized for examination of the biological material of interest. FIG. 5A shows a test cell comprising a focal plane 60 that is parallel to the flow 62 of the biological material through the channel 24 of the cell. In one embodiment, a parallel focal plane may be used where the channel is sufficiently narrow for the microscope to be focused at the center of the channel and still have a majority of the cells in focus. The channel may have a height 30 such that the diameter of the cells or parts of cells 64 may be slightly smaller than the height of the channel. In this way, the cells 66 flow by the objective lens at a depth of about one cell at a time, and the microscope focal plane may be parallel to the channel.

However, having the channel height 30 only slightly smaller than the average cell diameter may cause the channel to become clogged with larger cells or other components of the biological material. Also, a small channel size can result in lengthening of the time required for all of the cells to pass through the channel. Thus, in some cases, a larger cross-section channel may be desired. FIG. 5B shows a positioning of the objective lens of a microscope to provide for a focal plane 61 that is angled compared to the channel 24 and the flow 62 of the biological material. In this way, the top of the channel is in focus at one side of the focal plane and the bottom of the channel is in focus at the other side of the focal plane. This orientation may be preferred where the cell diameter 64 is substantially less than the height 30 of the channel. Where cells are flowing through a larger channel, the cells may be positioned at the top of the channel 67, the middle of the channel 68, or at the bottom of the channel 69. Using an angled focal plane 61, each cell flowing in the channel will be in focus as the cell crosses the focal plane, regardless of the flow path taken by any one cell.

The flow of the biological material past the objective lens may be varied depending upon the type of cells that are being examined. If the flow is slow, direct visual inspection of the cellular material may be possible. However, in most cases, the image may be filmed or a digital imaging technique may be applied. The rate that the biological material passes through the focal plane may depend upon the imaging technique employed. In alternate embodiments, a digital camera used with the methods, devices, and systems of the present invention may range from having a maximum rate of 30 frames per second (fps) to 100 fps, to 300 fps, to 1,000 fps. Thus, in alternate embodiments, where the width of the field of view is about 0.5 mm, a flow that may allow for evaluation by digital or machine analysis may range from about 15 mm per second to about 500 mm per second. In one embodiment, with a channel width of 0.5 mm and a height of 10 microns (μm), and filming the flow with a 30 fps camera, a specimen of 1 cc (cubic centimeter) may pass through the focal plane in about 13,300 seconds (i.e., about 4 hours). With a 300 frame per second camera, a 1 cc sample may be viewed in less than 20 minutes. Different rates of flow of the biological material through the focal plane may be used according to the specific viewing device and imaging device employed.

In one embodiment, the light used to image the cells may be flashed in sync with the frame rate of the camera to avoid blurring of the image due to movement of the cellular material. A stroboscopic light source may be used to produce one flash per frame. In an embodiment, where separation of the cells of interest from other components of the biological material has not been performed, a computer based machine vision program may be used to separate the images of the cells of interest from other components of the biological material. For example, 1 cc of semen may include several million sperm in addition to relatively few prostate cells.

In one embodiment, the device may combine the separation step with examination of the cells. For example, the cells or cell parts may be examined by causing a liquid biological material to rotate in a chamber under a microscope objective, and inspecting the cellular material with the microscope trained at the side of the chamber, where one of the chamber walls is used as a window for the microscope. To separate the cells from the remainder of the biological material, the biological material may be rotated in the chamber and the cells in the liquid pushed by centrifugal forced towards the walls of the chamber. The microscope may be focused to a point just inside of the chamber wall such that the cells that are traveling on the surface of the chamber wall pass through the focal plane of the lens.

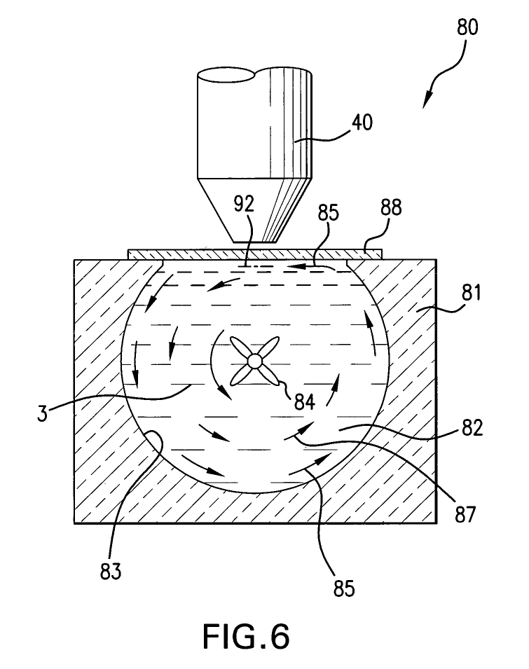

FIG. 6 shows an embodiment of a test device 80 where a biological material 3 comprising a liquid is rotated by a rotor 84 positioned in the center of a chamber 82. In FIG. 6 the test device 80 is shown from the top view. The microscope objective 40 may be positioned horizontally, so as not to create an air bubble at the cover glass 88. As described for the in-line flow cell, the cover glass may comprise flat parallel surfaces. A standard microscope cover glass may be used. Thus, the chamber 82 may be generally circular, but with a flat surface formed by the cover glass. As described for the in-line flow cell, the test device 80 may allow for the sample in the focal plane of the viewing device to be illuminated from any angle. For example, the chamber walls 81 may be made of glass or quartz. Or, the chamber walls may be made of a plastic, such as polystyrene, polycarbonate, polyethylene, polypropylene, or the like. Or the chamber walls may be made of a mineral or a ceramic such as fused quartz. Or, when illumination from the direction of the viewing device is used, the chamber walls may be made of a nontransparent material. In one embodiment a metal, such as stainless steel, may be used.

As the rotor spins, cells present in the biological material 3 may be pushed by centrifugal force to the side 83 of the chamber 82. The rotation of the rotor may then force the cellular material at the side 83 of the chamber to flow 85 through the focal plane 92 at the internal surface of the cover glass 88. Depending on the specific weight of the cells as compared to other materials in the liquid, the cells of interest may be the outer layer of the flow 85, or an inner layer 87, and the microscope may be focused on the appropriate layer.

The conditions for rotating the rotor 84 may be varied such that the cellular material of interest is positioned at the wall of the chamber. In one embodiment, the centrifugal force applied to the biological material in the chamber may be sufficient to force all of the cells to the surface of the chamber. Alternatively, the force may be such that only the heavier cells are pushed to the chamber wall, whereas lighter cells (or cellular substructures) are localized closer to the center of the chamber.

Dimensions of the device 80 for real-time separation of the cells from the remainder of the biological material may be as required for analysis of the biological material of interest. For example, the test cell may be sized to contain a single volume of reconstituted biological material as the analyte. In alternate embodiments, for smaller volumes, the chamber 82 may comprise a volume in the range of from about 50 microliters (μl) to about 20 ml, or from about 100 μl to about 5 ml, or from about 200 μl to about 2 ml. For larger volumes of the biological material (e.g., such as for examination of serum or a flushing solution), the correspondingly larger dimensions for the chamber 82 may be used. For example, for a larger volume of biological material, the chamber 82 may comprise a volume in the range of from about 2 ml to about 500 ml, or from about 5 ml to about 100 ml, or from about 10 ml to about 20 ml.

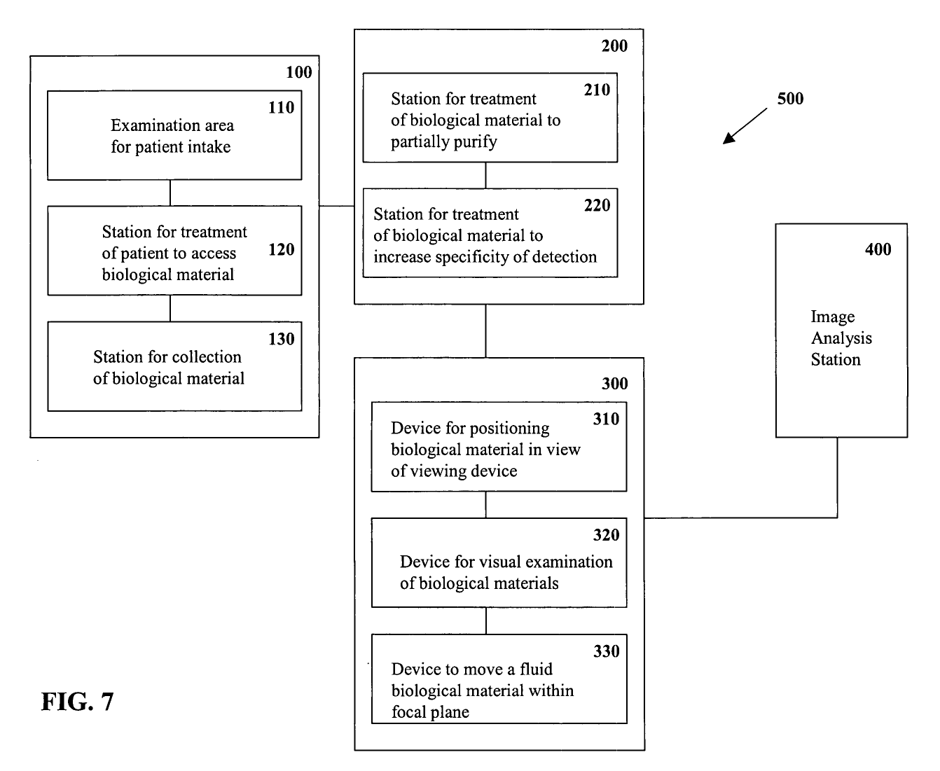

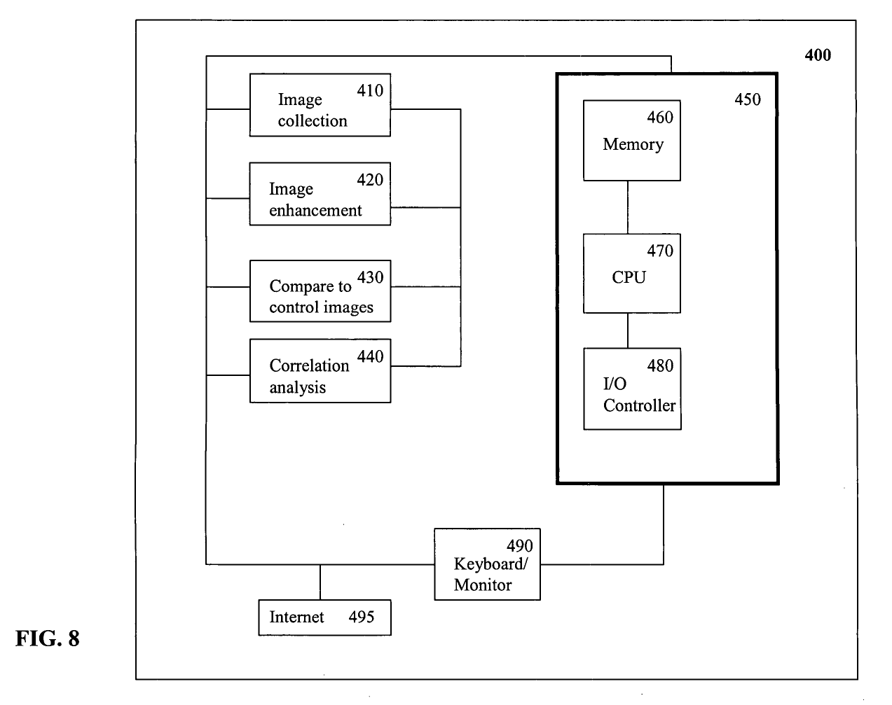

The present invention may also comprise a system 500 for examining a biological material for the presence of cellular material comprising at least one molecular characteristic correlated with cancer. FIGS. 7 and 8 shows an embodiment of a system of the present invention. The system of FIGS. 7 and 8 comprises several subsystems or stations, each of which, as a whole, or as parts thereof, may in itself comprise a system of the present invention. In an embodiment, the system may comprise a station 100 for sample procurement and the examination of patients. The station for sample procurement and patient examination may comprise an area for patient intake 110. The station for patient intake may comprise a database for the entry of data regarding the patient's medical history and any symptoms that the patient may be experiencing. The station for patient examination may also comprise a station to perform any procedures required to access the biological material of interest 120. For example, the patient may be induced to secrete milk from a lactating breast by pumping the breast. Or the patient may be induced to secrete seminal fluid. In another embodiment, the patient's urine may be collected. Or, the patient's intestinal tract may be flushed to remove cells from the surface of the intestines. The station for patient examination may further comprise a station for the collection of the biological material of interest 130. The station for the collection of biological material may comprise a means for labeling, storing, and categorizing the biological material until such time that the material may be analyzed. Upon collection, the sample may be treated so as to preserve the biological material for further analysis. For example, as described above, external agents such as a preservative, a buffering agent, or an agent to prevent coagulation may be added to the collected biological material. Such chemicals may include a physiological buffer (e.g., Tris buffer, citrate buffer, phosphate buffer) ethylenediamine tetraacetic acid (EDTA), EGTA, a salt or a sugar to adjust the osmolarity, an anti-oxidant, or other preservatives.

In an embodiment, the system may further comprise a station for treatment of the collected biological material of interest 200. In some cases it may be necessary to treat the biological material to at least partially purify the material 210. For example, it may be necessary to concentrate the cellular material. If the biological material has relatively few cells in a large volume of liquid, several processes may be used to concentrate the cells and reduce the volume of the liquid before examining the cellular material. The biological material may be subjected to a centrifugal force and cells collected and resuspended for further analysis. Or the biological material may be subjected to filtration through a filter of defined pore size to concentrate the cells and/or separate out cells and macromolecules from smaller diluents such as water.

The system may comprise a station where the biological material is treated to increase the specificity of detection 220. For example, the material may be exposed to an agent that may tag (i.e., label) the cells of interest. In an embodiment, the biological material may be treated to enhance the imaging of abnormal (e.g., pre-cancerous or cancerous) cells. For example, the biological material may be exposed to an antibody that recognizes a cell-specific membrane antigen. In this way, the cells of interest may be preferentially selected from a background of other cell types or cellular material. Or, the biological material may be exposed to an antibody that can bind to a protein expressed on the surface of a cancer cell. For example, cells may be exposed to an antibody to epidermal growth factor receptor (EGFR), a protein reported to be expressed in high levels in primary breast cancer. Or, the cells may be exposed to an agent that interacts with the cells, where the agent comprises a fluorophore. The fluorophore may be used to image the cells, or for pneumatic separation of the cells from other components of the biological material.

In an embodiment, the system may further comprise a station for examination of the biological material of interest 300. The station for examination of the biological material may comprise a device 310 for positioning the biological material for examination by a viewing device 320. The viewing device selected for use with the devices of the present invention may depend on the biological material being examined. In one embodiment, the viewing device may comprise a standard compound (or simple) microscope. Additionally or alternatively, the viewing device may comprise a standard film camera, a digital camera, or a movie camera. Both a still camera and a movie camera may use film or a digital detector such as a CCD or a CMOS detector. A film may be scanned after development by a human or, for a digital film, by a computer. Also, optical enhancement devices such as filters, or spatial filtering may be used to facilitate detection of abnormal cells by either a human or a computer program.

For examination of the cells by a viewing device, the cells may be separated from liquids and other components of the biological material in a centrifuge, spread on a solid substrate (e.g., a microscope slide), and the biological material viewed as a stationary field under a microscope. For example, a secreted biological material may be smeared on a substrate and examined for an abnormal morphology under a microscope. Or, cells from a cell pellet may be smeared on a substrate and examined for an abnormal morphology under a microscope. Alternatively or additionally, the cells may be stained to allow for a cytological analysis using cytochemical or immunohistochemical stains known in the art. To allow for sufficient sampling of the biological material, a plurality of samples may be viewed.

In an alternate embodiment, an in-line flow microscope may be used for examination of a liquid biological material by causing the liquid to flow in a narrow channel under a microscope objective. An in-line flow microscope may comprise an in-line flow cell, where the liquid secreted from the gland including the cells is made to flow in a narrow gap under a microscope's objective. Thus, the examination station 300 may further comprise a device 330 to cause a fluid biological material to move past the viewing device. For example, the system may comprise a flow inducer to apply positive pressure to the first chamber to thereby push a biological material through the focal plane.

In an embodiment, the images of the cellular material may then be sent to a station where the data may be collected and analyzed 400 (FIGS. 7 and 8). In one embodiment, the station for data analysis may comprise a device for image collection 410. The image of the suspected cells may be displayed for human observation and analysis of the image. If the imaging is done on stationary preparation of cells, then a still image can be sufficient. If the imaging is done on a flow of liquid, a series of images may be obtained. Or, machine vision tools may be employed. For a fast flow, the flow may be filmed with a movie camera for subsequent human visual inspection. For example, the camera may collect images of the cells as the biological material passes through the focal plane of a microscope. The machine vision technology may be preferred where the number of cells is quite low in a large volume of a liquid sample. The machine vision technology may also be used where the total number of cells is too large for direct analysis, or where the cells of interest are mixed with a large number of unrelated cells. In an embodiment, the images may then be processed by the imaging system 410.

In an embodiment, information from images obtained by examination of the biological material of interest may be collected to enhance resolution of the image of each cell. In one embodiment, the station for data analysis may further comprise a device for image enhancement 420. For example, image processing technology may be used to sharpen the images of cells.

Also, the data may also be processed to remove noise 420. In some cases, the user, via the keyboard 490, floppy disk, or remote access (e.g., internet or intranet) 495, may want to input variables or constraints for the analysis, as for example, the threshold for determining noise.

Image analysis programs may be used to estimate parameters of the cells such as the diameter of the cell's nucleolus, the shape of the cell, and other variables that may comprise a molecular characteristic correlated with cancer. In one embodiment, a two-dimensional cross-correlation technique and/or two-dimensional transforms may be used to compare cells to known benchmarks of cancerous cells.

A final decision may be taken by a computer program or by a human after examining the images of suspected cells. In an embodiment, the analysis station 400 may be equipped with a computer 450 that runs a computer program that is able to recognize the cells or cell parts of interest and determine whether the cell is abnormal. Criteria for cells suspected for malignancy may be developed. For example, in one embodiment, abnormal cells may comprise an unusual shape. Alternatively or additionally, abnormal cells may comprise an unusual staining with antibody to a cell-specific protein, or an unusual profile of sub-cellular structures.

In one embodiment, the computer program can process the collected images and select the images that are specific to the cell type of interest. Thus, in an embodiment, the computer program (i.e., programming code) may comprise a plurality of steps whereby a visual image of each of a plurality of cells are compared to an image representing other components of the biological material. For example, prostate cells in seminal fluid may be distinguished from sperm or non-prostate cells that may be present. Alternatively or additionally, the computer program may comprise a series of steps whereby the images of the cells are collected and compared to an image representing a normal (i.e., not cancer) cell and an abnormal (i.e., cancer) cell 430. The computer program may also include an analysis of the images 440. If the image comprises a significant overlap with the cancer cell image, there may be a selection step whereby the image is classified as being an abnormal cell. If the image comprises a significant overlap with a normal image, the image may be accorded the status of normal or untransformed tissue.

The images may be saved for further review. In an embodiment, there may be a criterion applied such that if pre-determined number of images are registered as abnormal, the patient sample is considered abnormal. Where a patient sample is accorded the status of abnormal, the patient may be contacted for a follow-up examination.

In an embodiment, once the data has been collected (i.e., using the imaging system or other type of data collection system), it may be compiled and/or transformed if necessary by statistical analysis. Thus, in one embodiment, the image comparison and correlation analysis 430, 440 comprise statistical techniques. Statistical techniques such as principle components analysis (PCA), Bayesian Networks, or cluster analysis may be used. Such analysis techniques are all widely available in packaged software.

For example, cluster analysis techniques, such as K-Means or self-organizing maps (SOM), or other types of grouping techniques principal components analysis (PCA) may be used. Cluster analysis is a loose term covering many different algorithms for grouping data. Clustering can be divided into two main types: top-down and bottom-up. Top-down clustering starts with a given number of clusters or classes and proceeds to partition the data into these classes. Bottom-up clustering starts by grouping data at the lowest level and builds larger groups by bringing the smaller groups together at the next highest level.

For example, K-Means is an example of top-down clustering. K-means groups data into K number of best-fit clusters. Before using the algorithm, the user defines the number of clusters that are to be used to classify the data (K clusters). The algorithm randomly assigns centers to each cluster and then partitions the nearest data into clusters with those centers. The algorithm then iteratively finds new centers by averaging over the data in the cluster and reassigning data to new clusters as the centers change. The analysis iteratively continues until the centers no longer move (Sherlock, G., Current Opinion in Immunology, 12:201, 2000).

Self-organizing maps (SOMs), a type of bottom-up cluster analysis, are competitive neural networks that group input data into nearest neighbors (Torkkola, K., et al., Information Sciences, 139:79, 2001; Toronen, P., et al., FEBS Letters, 451:142-146, 1999). As data is presented to the neural network, neurons whose weights currently are capable of capturing that data (the winner neuron) are updated toward the input. Updating the weights, or training the neural net, shifts the recognition space of each neuron toward a center of similar data. SOMs are similar to K-means with the added constraint that all centers are on a 1 or 2 dimensional manifold (i.e., the feature space is mapped into a 1 or 2 dimensional array, where new neighborhoods are formed).

Principal component analysis (PCA) (see e.g., Jolliffe, I.T., Principal Component Analysis, New York: Springer-Verlag, 1986) is a stepwise analysis that attempts to create a new component axis at each step that contains most of the variation seen for the data. Thus, the first component explains the first most important basis for the variation in the data, the second component explains the second most important basis for the variation in the data, the third component the third most important basis, and so on. PCA projects the data into a new space spanned by the principal components. Each successive principal component is selected to be orthogonal to the previous ones, and to capture the maximum information that is not already present in the previous components such that linear combinations of the principal components (eigenarrays) compose the original data. These principal components are the classes of data in the new coordinate generated by PCA. If the data is highly non-correlated, then the number of significant principal components can be as high as the number of original data values.

In another embodiment, Bayesian networks are used to determine the probability that the sample comprises cancer cells based on the physiological parameter being ascertained. The Bayesian network analysis may further comprise the use of orthologous data analysis (see e.g., Hoque et al., J. Clin. Oncol., 23:6569-6575, 2005).

Also, the data may be tabulated using a spreadsheet software such as Microsoft Excel, FoxPro, Lotus, or the like. In an embodiment, the data are entered into the system for each experiment. Alternatively, data from previous runs may be stored in the computer memory 460 and used as required.

At each point in the analysis, the user may input instructions via a keyboard 490, floppy disk, remote access (e.g., via the internet) 495, or other access means. The user may enter instructions including options for the run, how reports should be printed out, and the like. Also, at each step in the analysis, the data may be stored in the computer 450 using a storage device common in the art such as disks, drives or memory 460. As is understood in the art, the processor 470 and I/O controller 480 are required for multiple aspects of computer function. Also, in an embodiment, there may be more than one processor.

It will be understood that each of the elements described above, or two or more together, may also find utility in applications differing from the types described. While the invention has been illustrated and described as methods, devices, and systems for detection of cancer in a biological material from an organ, it is not intended to be limited to the details shown, since various modifications and substitutions can be made without departing in any way from the spirit of the present invention. As such, further modifications and equivalents of the invention herein disclosed may occur to persons skilled in the art using no more than routine experimentation, and all such modifications and equivalents are believed to be within the spirit and scope of the invention as described herein. All patents and published patent applications referred to in this document are incorporated by reference in their entireties herein.

That which is claimed is:

1. A method for testing a subject for the presence of cancer cells in an organ in a subject comprising the steps of:
(a) accessing a biological material from the organ by causing the biological material to be emitted from the organ, wherein the biological material comprises a fluid suspension of a plurality of cells or parts of cells from the organ;
(b) collecting the biological material; and
(c) examining the biological material for cells comprising at least one molecular characteristic correlated with cancer.

2. The method of claim 1, wherein the step of accessing the biological material comprises causing the biological material to be secreted from an organ.

3. The method of claim 1, wherein the step of accessing the biological material comprises causing the biological material to be dislodged from an organ.

4. The method of claim 3, wherein the step of dislodging the biological material comprises applying massage or vibrations to the organ.

5. The method of claim 3, wherein the biological material is dislodged from the organ by flushing the organ with a liquid and collecting the liquid.

6. The method of claim 1, wherein the organ comprises at least one of a prostate, a pancreas, a liver, a mammary gland, a thyroid gland, an adrenal gland, a testis, a saliva gland, a gall bladder, an ovary, a bladder, an esophagus, a stomach, a duodenum, a small intestine, or a colon.

7. The method of claim 1, further comprising a step of at least partly separating a portion of the cells or cell parts from at least one other component of the biological material prior to the step of examining the cells or cell parts for at least one characteristic correlated with cancer.

8. The method of claim 7, wherein the step of at least partly separating the cells or cell parts from at least one other component of the biological material comprises subjecting the biological material to a centrifugal force.

9. The method of claim 1, wherein the step of examining the biological material for cells comprising at least one molecular characteristic correlated with cancer comprises viewing at least a portion of the biological material with a microscope.

10. The method of claim 9, further comprising causing the biological material to move past the lens of a microscope for optical examination.

11. The method of claim 10, further comprising causing the biological material to rotate in a chamber and examining at least a portion of the cells or cell parts that are forced to the outer surface of the chamber as the biological material moves past the lens of a microscope.

12. The method of claim 1, wherein the biological material is exposed to an agent that can specifically interact with a marker on the surface of a malignant cell.

13. A device for examining a biological material for the presence of cells comprising at least one molecular characteristic correlated with cancer, the device comprising a chamber having at least one portion that comprises a focal plane of a viewing device suitable for optical examination of the biological material.

14. The device of claim 13, wherein the chamber comprises a first reservoir for storing a first portion of the biological material prior to optical examination, and a channel for positioning a second portion of the biological material in the focal plane of a viewing device.

15. The device of claim 14, wherein the channel comprises a first end and a second end such that being examined, the biological material flows from the first end of the channel to the second end of the channel in a manner such that at least a portion of the flow comprises the focal plane of the viewing device.

16. The device of claim 14, wherein the first reservoir for storing the first portion of the biological material is positioned on the first end of the channel such that the biological material moves from the first reservoir into the channel.

17. The device of claim 14, further comprising a second reservoir for collecting the biological material after examination of the biological material by the viewing device.

18. The device of claim 17, wherein the second reservoir is positioned on the second end of the channel such that the biological material moves from the channel to the second reservoir.

19. The device of claim 13, wherein the chamber comprises a rotating part, such that when rotated, the rotating part pushes at least a portion of the biological material to the perimeter of the chamber, and wherein the perimeter of the chamber comprises the focal plane for the viewing device.

20. The device of claim 13, wherein the focal plane is parallel to a flow of the cells through the focal plane.

21. The device of claim 13, wherein the focal plane is angled to a flow of the cells through the focal plane.

22. The device of claim 13, wherein the viewing device comprises a microscope.

23. A system for examining a biological material for the presence of cells comprising at least one molecular characteristic correlated with cancer comprising:
(a) an examination device comprising a chamber having at least one portion that comprises a focal plane of a viewing device suitable for optical examination of the biological material;
(b) a viewing device for optical examination of the biological material in the focal plane; and
(c) a means to cause the biological material to move through the focal plane.

24. The system of claim 23, wherein the examination device chamber comprises a first reservoir for storing a first portion of the biological material prior to optical examination, and a channel for positioning a second portion of the biological material in the focal plane of a viewing device.

25. The system of claim 24, wherein the channel comprises a first end and a second end such that while the cells are being examined, the biological material flows from the first end of the channel to the second end of the channel in a manner such that at least a portion of the flow comprises the focal plane of the viewing device.

26. The system of claim 25, wherein the first reservoir for storing the first portion of the biological material is positioned on the first end of the channel such that the biological material moves from the first reservoir into the channel.

27. The system of claim 25, further comprising a second reservoir for collecting the biological material after examination of the biological material by the viewing device.

28. The system of claim 27, wherein the second reservoir is positioned on the second end of the channel such that the biological material moves from the channel to the second reservoir.

29. The system of claim 23, wherein the flow of the biological material is parallel to the focal plane.

30. The system of claim 23, wherein the flow of the biological material is at an angle to the focal plane.

31. The system of claim 23, wherein the optical examination comprises examination of the biological material under a microscope.

32. The system of claim 23, further comprising a computer-readable medium on which is encoded programming code for the analysis of the biological material, the programming code comprising a plurality of steps whereby a visual image of each of a plurality of cells are compared to a first image representing an untransformed cell and a second image representing an abnormal cell.
